(19) United States
(12) Patent Application Publication
(10) Pub. No.: US 2007/0038310 A1
(43) Pub. Date: Feb. 15, 2007

(54) Set for introducing a plastic surgery implant, a case for introducing such an implant and a corresponding production method

(76) Inventor: Richard Jean-Claude Guetty, Miribel (FR)

Correspondence Address: POWELL GOLDSTEIN LLP, ONE ATLANTIC CENTER, FOURTEENTH FLOOR 1201 WEST PEACHTREE STREET NW, ATLANTA, GA, 30309-3488, US

(21) Appl. No.: 10/565,011

(22) PCT Filed: Jul. 16, 2004
(86) PCT No.: PCT/FR04/01892

§ 371 Date: Jul. 25, 2006

(30) Foreign Application Priority Data
Jul. 18, 2003 (FR) 03/08808

(51) Int. Cl. A61F 2/02; A61F 2/12

(52) U.S. Cl. 623/23.72; 623/8

(57) ABSTRACT

The invention relates to a set for introducing a plastic surgery implant into patient body comprising a plastic surgery implant to be implanted into a patient body, said implant is embodied in such a way that it is deformable, thereby enabling it to change the shape thereof from a shape used for introduction into the body into a functional shape inside said body, a case which envelops said implant in the introducing shape thereof and is provided with an opening unit activateable by a positive action, thereby enabling it to change the closed shape thereof when it confines the implant in the introducing shape into an open shaped when it makes it possible to shape said implant in to the functional shape thereof.

### PRIORITY CLAIM

This patent application is the U.S. National Phase of International Application No. PCT/FR/2004/001892, having an International Filing Date of Jul. 16, 2004, which claims priority to French Patent Application No. 03/08808, having a Filing Date of Jul. 18, 2003, the disclosures of which are incorporated herein by reference in their entirety.

### FIELD OF THE INVENTION

This invention relates to the technical field of artificial devices, of the implant or prosthesis type, designed to restructure the shape of a part of the human body, for aesthetic and/or therapeutic purposes.

This invention more particularly but not exclusively relates to the field of mammary implants, designed to provide mammary reconstruction in the case of mastectomy, or to achieve mammary augmentations of an aesthetic character.

This invention relates to a kit for introduction of a plastic surgery implant into the body of a patient.

This invention also relates to a case for introduction of a plastic surgery implant into the body of a patient.

This invention relates, moreover, to a method for manufacture of a kit for introduction of a plastic surgery implant into the body of a patient.

This invention relates, finally, to a new use of a chain stitch.

### BACKGROUND OF THE INVENTION

Implantation of a foreign body in the body of a patient in order carry out reconstructive surgery is known, whether this is for the purpose of reconstruction of a part of the body, for example following an accident or illness, or for aesthetic purposes, to change the appearance of a part of the body.

In particular, implantation of an implant in the chest of a female patient in order to carry out a mammoplasty is known, for the purpose of reconstruction, for example following a mastectomy, and/or for aesthetic purposes, in order to augment the volume of the breast.

Implantation of such foreign bodies for purely aesthetic purposes is also known, for example in the buttocks, the legs, the arms or the pectorals.

These foreign bodies, which are generally designated by the name “prostheses” or “implants,” most often come in the form of a flexible pouch, made, for example, out of a biocompatible elastomeric material, and enclosing a certain quantity of a filler material, of the silicone gel or physiological saline type, which gives to the prosthesis its functional volume.

These implants are generally introduced subcutaneously, through an incision made in proximity to the final implantation zone of the implant.

In the specific case of mammary implants, these are generally introduced subcutaneously in the thorax of the patient, over or under the pectoral muscle, through an incision that can be made in proximity to the axillary space, along the sub-mammary cleft, or along the contour of the areola of the breast.

Such plastic surgery operations quite obviously generally present an aesthetic purpose; that is why it is paramount, especially in the case of a mammoplasty, to minimise the size of the incision allowing introduction of the implant, this in order to limit the presence of an unsightly post-operatory scar.

In order to do so, the surgeon generally makes an incision whose size is clearly less than the size of the implant in its functional state.

Introduction of the implant through this small incision is possible due to the flexible and deformable character of the implant, which makes it possible for the surgeon to model it according to a shape which allows its threading through the incision into the patient's body.

This introduction operation, which is carried out manually by the surgeon, generally presents for the latter a character that is relatively time-consuming and difficult because it requires difficult manipulations of compression and pushing on the implant in order to introduce it under the patient's skin.

In order to facilitate this introduction operation, we also know, moreover, to prepare a reception chamber for the implant by means of the implementation, prior to implantation of the implant, of a subcutaneous tissue retractor.

It is thus easier for the implant to go in through the incision since it doesn't have to create its housing for itself by spreading apart the tissues.

It is to be noted, however, that this way of proceeding involves the implementation of numerous surgical steps (incision, introduction of the retractor, dilation of the retractor, contraction of the retractor, explantation of the retractor, implantation of the prosthesis), which increases the duration, the cost and especially the trauma and the risk associated with this operation.

Moreover, mammary implants presenting an inflatable character are also known. Such implants come in the form of a flexible pouch partially filled with fluid, even completely empty. Inflation of the implant so that it attains its functional volume is carried by means of an inflation tube, once merely the implant is positioned under the skin of the patient. Such implants certainly make it possible to make an introduction incision presenting a size less than that of traditional non-inflatable implants, but their implantation presents, however, essentially the same difficulties as those described previously, namely the necessity for the surgeon to perform, difficult and time-consuming manipulations in which the practitioner must simultaneously shape the flexible pouch so that it may pass through the incision, while pushing on said pouch so that it goes in under the skin in the proper direction, avoiding any untimely crumpling or entanglement of the pouch which could hamper its subsequent inflation.

### SUMMARY OF THE INVENTION

The features of the present invention consequently aim at remedying the various disadvantages enumerated previously and in proposing a kit and a case for introduction of a plastic surgery implant into the body of a patient making possible the subcutaneous emplacement of a plastic surgery implant in a particularly simple, rapid and safe way.

Another feature of the invention aims at proposing a kit and a case for introduction of a plastic surgery implant into the body of a patient presenting a character that is particularly reliable and comfortable for the patient.

Another feature of the invention aims at proposing a kit and a case for introduction of a plastic surgery implant into the body of a patient presenting reduced size as well as good dimensional stability and uniformity.

Another feature of the invention aims at proposing a kit and a case for introduction of a plastic surgery implant into the body of a patient the construction of which presents a good compromise between weight/strength.

Another feature of the invention aims at proposing a kit and a case for introduction of a plastic surgery implant into the body of a patient that presents an atraumatic contact for the biological tissues.

Another feature of the invention aims at proposing a kit and a case for the introduction of a plastic surgery implant into the body of a patient the manufacture of which is particularly easy and rapid.

Another feature of the invention aims at proposing a manufacturing method for a kit for introduction of a plastic surgery implant into the body of a patient which is particularly simple and rapid to implement, while making it possible to produce a kit presenting excellent reliability.

Another feature of the invention aims at proposing a manufacturing method for a kit for introduction of a plastic surgery implant into the body of patient which makes it possible to produce a kit presenting a reduced size.

The features assigned to the invention are attained by means of a kit for introduction of a plastic surgery implant into the body of a patient comprising, in one exemplary embodiment:

a plastic surgery implant designed to be implanted in the body of a patient, said implant presenting a deformable character which makes it possible for it to pass from a configuration for introduction into the body to a functional configuration within the body,

a case shaped to envelope said implant in the introduction configuration, said case being provided with an opener member that can be activated by positive action making it possible for it to pass on the one hand from a closed configuration, in which it confines implant in its introduction configuration to, on the other hand, an open configuration, in which it enables deformation of said implant into its functional configuration,

case comprising a locking means, linked functionally to the opener member and making it possible to immobilise by itself, without any external action on said means, case in the closure configuration, said kit being characterised in that case is provided with a thread having a first portion sewn as a single-thread chain stitch so as to form said locking means, and having a second portion that remains free and forms the opener member, actionable by traction.

The features of the invention are also attained by means of a case for introduction of a plastic surgery implant into the body of a patient, said implant presenting a deformable character making it possible for it to pass from a configuration for introduction into the body to a functional configuration within the body, said case being shaped to envelope said implant in the introduction configuration and being provided with an opener member that can be activated by positive action making it possible for it to pass, on the one hand, from a closed configuration, in which it confines implant in its introduction configuration to, on the other hand, an open configuration, in which it enables deformation of said implant into its functional configuration, said case comprising a locking means linked functionally to opener member and making it possible to immobilise by itself, without any external action on said means, case in the closure configuration, said case being characterised in that it is provided with a thread having a first portion sewn as a single-thread chain stitch so as to form said locking means, and having a second portion, that remains free and forms the opener member, actionable by traction.

The features of the invention are also attained by means of a manufacturing method for a kit for introduction of a plastic surgery implant into the body of a patient in which:

a plastic surgery implant is supplied or manufactured, said implant presenting a deformable character making it possible for it to pass from a configuration for introduction into the body to a functional configuration within the body,

a case is supplied or manufactured, designed to envelope said implant in the introduction configuration, said case essentially presenting, when it is in the closed configuration, the shape of a sheath,

said method being characterised in that it comprises a step for insertion of the implant into the sheath in which:

the implant is shaped in the introduction configuration,

then the implant is progressively constrained along its length by means of a jig, so as to reduce the cross-section of said implant, while simultaneously covering the implant with the sheath in the closed configuration.

The features assigned to the invention are also attained by means of the use of a chain stitch in accordance with class 101 of the NF G 05-002 standard of December 1982 as locking means for a case for introduction of a plastic surgery implant into the body of a patient.

### BRIEF DESCRIPTION OF THE DRAWINGS

Other features and advantages of the invention will appear better upon reading of the following description, as well as by means of the appended drawings, given in a purely illustrative and non-restrictive way, in which:

FIG. 1 shows, in perspective view, a case for introduction of a plastic surgery implant into the body of a patient in accordance with the invention.

FIG. 2 shows, in a sectional side-view, a detail of the construction of the locking means for a case in accordance with a preferential exemplary embodiment.

FIG. 3 shows, in a bottom view, the construction detail shown in FIG. 2.

FIG. 4 shows, schematically, the principal steps of the manufacturing method in accordance with the invention, according to a first specific mode of implementation.

FIG. 5 shows, in a perspective view, a detail of a specific exemplary embodiment of the introduction case in accordance with the invention.

FIG. 6 shows, schematically, a second specific mode of implementation of the method in accordance with the invention.

FIG. 7 shows, schematically in a top view, a first example of construction of a mammary implant in accordance with the invention.

FIG. 8 shows, in a side view, the implant of FIG. 7.

FIG. 9 shows, schematically in a top view, a second example of construction of a mammary implant in accordance with the invention.

FIG. 10 shows, in a side view, the implant of FIG. 9.

FIG. 11 shows, schematically in a top view, a third example of construction of a mammary implant in accordance with the invention.

FIG. 12 shows, in a side view, the implant of FIG. 11.

### DESCRIPTION OF THE INVENTION

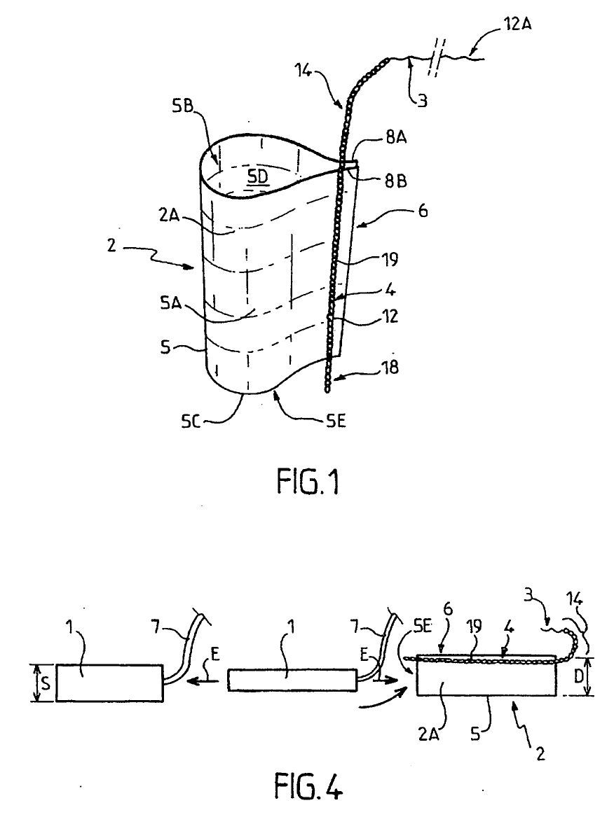

FIGS. 1 to 6 show a kit for introduction of a plastic surgery implant 1 into the body of a patient, as well as the details of its construction.

By “plastic surgery,” we designate in what follows surgery intended to modify the shape of an organ, or of a part of the body, in order to correct a congenital or acquired anomaly, and/or in order to modify the aesthetics of the patient's body, in the sense for example of an increase in volume.

Plastic surgery comprises in particular a branch designated “aesthetic surgery” which is concerned principally with the remodelling of parts of the body for purposes that are essentially aesthetic.

This invention is therefore a matter for plastic surgery, and preferentially of aesthetic surgery.

The kit in accordance with the invention is designed to make possible the insertion under the patient's skin of an implant 1 to reconstruct and/or remodel and/or increase the volume of a part of the body, for example a breast, insofar as said implant, which presents a pre-determined functional volume, increases or replaces biological tissue, for example mammary tissue.

The kit in accordance with the invention is preferentially designed to make possible implantation through an incision previously made by the surgeon.

In the specific case of mammary implants, the kit in accordance with the invention can be adapted in particular to implantation by the axillary, sub-mammary, peri-areolar or trans-areolar route.

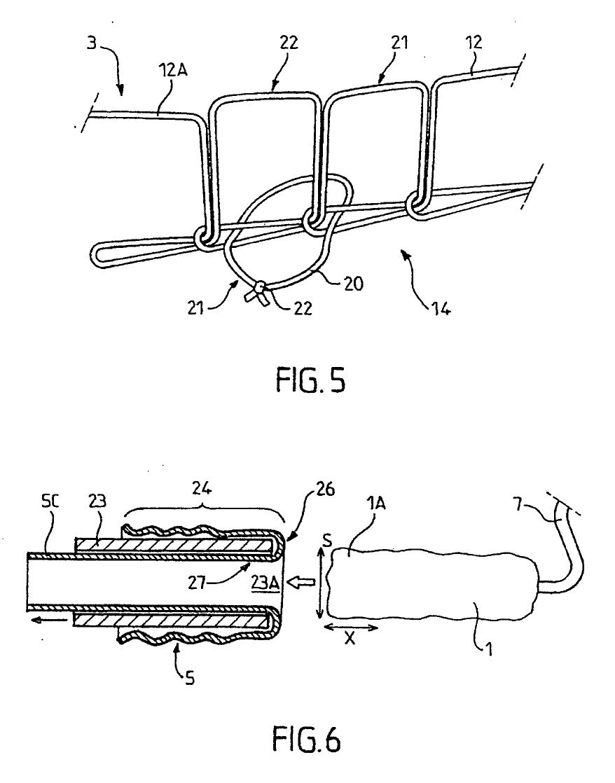

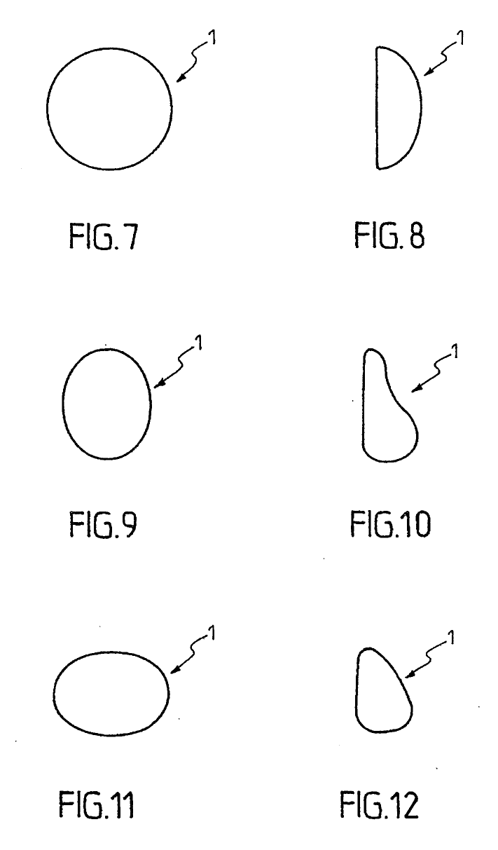

In accordance with the invention, the introduction kit comprises a plastic surgery implant 1 designed to be implanted in the body of a patient, said implant presenting a deformable character that makes it possible for it to pass from a configuration of introduction (shown schematically in FIGS. 4 and 6) into the body to a functional configuration within the body (shown in FIGS. 7 to 12, in the case of a mammary implant).

Plastic surgery implants are well known to those skilled in the art.

By way of example, the plastic surgery implant 1 can be an implant for the arm (forearm, biceps), of the leg (calf, thigh), of the buttocks, or of the chest (mammary implant for women, pectoral implant for men).

The invention is particularly well adapted to mammary implants.

The configuration for introduction of the implant corresponds to a conformation of the implant making it possible for the latter to pass through the introduction incision made beforehand by the surgeon.

This introduction configuration can be obtained by constraining implant 1 to adopt a shape with reduced transverse section S of a dimension essentially close to or less than that of the incision.

In the case in which implant 1 presents an expandable character, i.e. when, for example, it is designed to be inflated by a fluid once positioned in the patient's body, the introduction configuration will correspond to a deflated state of implant 1.

Preferentially, the plastic surgery implant 1 in introduction configuration presents an elongated shape that is essentially unidirectional or oblong such as shown in FIGS. 4 and 6.

As to the functional configuration, it corresponds to the active state of implant 1, i.e. the state in which the implant presents a pre-determined functional volume designed to effectuate reconstruction and/or tissue augmentation. Implant 1 therefore generally presents in its functional configuration a volume, or at least a size, greater than that which it presented in the introduction configuration.

As shown schematically in FIGS. 7 to 12, in the case in which implant 1 is a mammary implant, the latter may present, in the functional configuration, varied shapes, said shape being chosen by the surgeon and the patient according to surgical and/or aesthetic criteria.

FIGS. 7 and 8 thus show a mammary implant of the “spherical” type, presenting the general shape of a segment of a sphere.

FIGS. 9 to 12 show mammary implants of the “water drop” type, whose general shape is essentially anatomical.

Implant 1 in accordance with the invention may in particular present a general form that is convex (FIGS. 11 and 12) or concave (FIGS. 9 and 10), as well as a surface state that is textured or not.

The deformable character of implant 1 in accordance with the invention corresponds to the facility that the implant possesses, intrinsically or under the effect of external applied inflation forces, to pass from its introduction configuration to its functional configuration.

This deformable character therefore corresponds to an ability to deploy, said deployment being caused for example by a shape memory effect, an elastic effect, an inflation, or a combination of several of these characteristics.

In a general and standard way, implant 1 will present a flexible and pliable character.

In what follows, reference will more particularly be made, in, however, a non-restrictive way, to a plastic surgery implant 1 comprising at least one flexible pouch defining a predetermined internal volume, said at least one flexible pouch being equipped with connection means arranged to receive a connection unit 7 designed to be linked to a fluid source, for the purpose of carrying out the expansion of said pouch in the patient's body by filling with said fluid (for example a saline solution or a silicone gel, even a gas).

Said pouch is more preferably constructed in a standard way with a base of flexible materials, of the elastomeric type (such as silicone).

To best advantage, the connection means include an orifice and a valve, to detachably receive the connection unit 7, which is for example a catheter.

Said catheter 7 is linked to the fluid source (gas or liquid for example), which is located outside the patient's body, for the purpose of handling the expansion, i.e. the inflation, of said pouch in the patient's body by filling with fluid.

In a preferential variant (not shown), the plastic surgery implant (1) comprises on the one hand at least one first pouch and on the other hand at least one second pouch of volume less than the first pouch and arranged inside said first pouch, said first and second pouches being designed to enclose, respectively, a first and a second filling material, said first and second filling materials possibly presenting different densities. To best advantage, said second pouch is arranged in a way essentially concentric to the first pouch.

The introduction kit in accordance with the invention also comprises a case 2 shaped to envelope the plastic surgery implant 1 in the introduction configuration.

Case 2 is designed to contain and sheath implant 1 in the introduction configuration so as to form with said implant a streamlined whole, compact and with a surface that is regular overall, in order to facilitate introduction into the patient's body.

Case 2 thus constitutes a case for introduction of plastic surgery implant 1. This means that case 2 is designed and proportioned to answer to the specific technical and medical requirements linked to the subcutaneous placement of a plastic surgery implant, of the mammary implant type.

In the specific case in which the plastic surgery implant 1 is a mammary implant, case 2 is designed and proportioned to answer to the specific technical and medical requirements linked to the placement of such a mammary implant.

More particularly, case 2 is itself designed to be partially or totally introduced under the patient's skin, in such a way as to bring implant 1 to its final subcutaneous emplacement. The characteristics of case 2 (atraumatic, sterile character, etc.) are therefore quite obviously adapted to this introduction, certainly temporary, within the patient's body.

The general principle of the invention therefore rests on a covering of the implant according to a streamlined shape facilitating subcutaneous introduction. Case 2 thus serves as a vehicle for transport within the patient's body.

In accordance with the invention, case 2 is equipped with an opener member 3 that can be activated by positive action making it possible for it to pass, on the one hand, from a closed configuration (shown in particular in FIGS. 1 and 4), in which it is capable of confining implant 1 in its introduction configuration to, on the other hand, an open configuration (not shown in the figures), in which it makes possible the deformation of said implant 1 into its functional configuration; i.e. it essentially liberates implant 1 from any interaction preventing its deployment.

By “can be activated by positive action,” we are here designating the fact that the opener member 3 can be activated on command, for example by the physician carrying out the implantation, as opposed to passive opener members, which are for example activated by deployment of implant 1.

To best advantage, case 2 comprises a locking means 4 that make it possible to immobilise by itself, without any external action on said means 4, case 2 in the closure configuration.

In other words, locking means 4 locks case 2 in the closure configuration, without an external support intervention of this locking, for example applying tension, being necessary.

By “locking ” we are designating in particular here the fact that case 2, even under the effect of external forces tending to cause it to evolve into its open configuration, will remain immobilised in the closed configuration.

Locking means 4 is functionally linked to the opener member 3, in such a way that when the opener member 3 is activated, it cancels the effect of locking means 4, which makes it possible for case 2 to pass into the open configuration.

Thus, case 2 cannot pass from its closed configuration to its open configuration except when the opener member 3 is activated, and remains insensitive to any other possible external applied force.

Case 2 in the closed configuration is thus, by its construction, naturally locked in the closure position.

To best advantage, case 2 comprises a sheath 5, presenting a shape that is essentially tubular. Sheath 5 is delimited by a lateral envelope 5A extending between a proximal end 5B and a distal end 5C. In the exemplary embodiment shown in FIG. 1, the sheath comprises an axial opening 5E, 5D at each of its distal and proximal ends. For all of this it is altogether conceivable, without leaving the framework of the invention, that sheath 5 could be closed at one of its ends, more preferably at its proximal end 5D, or even at its two ends.

To best advantage, proximal end 5B of the sheath is assembled, for example by gluing, to the connection unit 7 linked to implant 1, in such a way that when the connection unit 7 is removed from the patient's body once the implantation has been carried out, it carries along with it case 2 so as to leave only implant 1 inside the patient's body.

Sheath 5 is fitted with at least one lateral opening 6 arranged over all or part of its length. Said lateral opening 6 may extend, as shown in FIG. 1, essentially longitudinally over the length of sheath 5. It is altogether conceivable, without on this account leaving the framework of the invention, that this lateral opening 6 might extend in any other way, and for example transversally or helically. Said lateral opening 6 is closed by locking means 4 when case 2 is in the closed configuration (FIG. 1), said lateral opening 6 being opened up, in order to make possible the deformation of implant 1 into its functional configuration, when case 2 is in the open configuration (not shown).

Preferentially, sheath 5 is split over all or part of its length, said split constituting lateral opening 6.

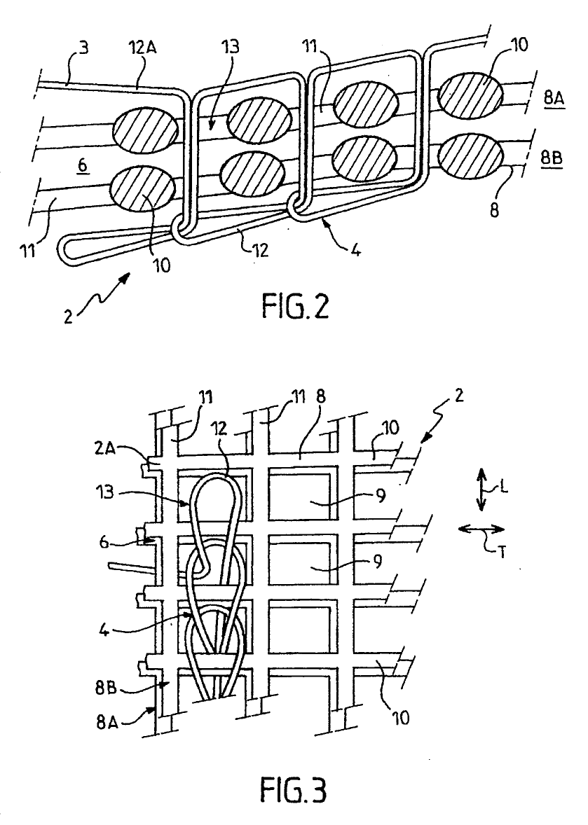

To best advantage, sheath 5 is formed from a textile lattice 8, as more particularly shown in FIGS. 2 and 3. The textile lattice 8 is formed from a network of crossed weft 10 and warp 11 threads which delimit empty zones 9, in such a way that the lattice presents an openwork structure.

Lattice 8 may be produced by any method well known to those skilled in the art, and for example in a way that is woven or not, braided or knitted.

Preferentially, the network of weft 10 and warp 11 threads is produced with a crossing of the warp and weft threads at 90° (square construction), as shown in FIG. 3.

To best advantage, sheath 5 is formed from a textile lattice 8 whose two opposite edges 8A, 8B are interlocked by locking means 4, in such a way that lattice 8 is shaped in an essentially tubular fashion.

In the exemplary embodiment shown in FIG. 1, sheath 5 is thus made up of a textile lattice 8 presenting a general form that is essentially rectangular, with four sides parallel two by two. Sheath 5 is produced by joining two opposite sides.

Preferentially, textile lattice 8 is constructed by knitting of polyester multifibre threads. It is, however, altogether conceivable to use other types of threads and in particular threads made of monofibre polyester, monofibre or multifibre polypropylene, or of cotton, cellulose, silk.

In a general way, it is particularly desirable in the context of the invention to use a lattice 8 of the type used in the context of the manufacture of parietal reinforcement plates, such as those implemented for the treatment of hernias or ruptures for example.

Resort to a textile lattice is, however, not all obligatory in the context of the invention, and it is possible to conceive of constructing case 2 from any other structure, and for example from a continuous structure (non-openwork) such as a fabric (more preferably woven very tightly and of low thickness), or a membrane made of plastic material.

In particular, in a preferential exemplary embodiment, sheath 5 is formed from a fabric whose two opposite edges are interlocked by locking means 4, in such a way that the fabric is shaped in an essentially tubular way. This fabric is to best advantage constructed by the weaving of threads principally based on polyamide, of the nylon® thread type.

By way of example, sheath 5 can be constructed of an essentially rectangular piece of commercial parachute cloth, such as RIP STOP® cloth manufactured by Tyco Plastics, weaving of 6.6 high tenacity polyamide threads.

More preferably, the cloth used will undergo operations of cleaning and scouring, in order to eliminate any possible toxicity.

To best advantage, sheath 5 is constructed from a material that is flexible but essentially non-elastic. In other words, the material making up sheath 5 is chosen to present an ability to be folded or rolled, while presenting a certain longitudinal and transverse rigidity, after the fashion of a sheet of paper.

Thus, in the case in which the sheath is a textile lattice 8 or a fabric, the latter present only little or no capability for extension in the longitudinal direction L and the transverse direction T, which does not prevent the lattice 8 or the fabric from presenting a certain intrinsic flaccidity.

In this case, sheath 5 presents essentially no capability to stretch radially.

It is, however, altogether conceivable, without on this account leaving the context of the invention, that case 2 might be constructed from a material presenting an elastic character, such as a biocompatible silicone polymer.

To best advantage, at least part of surface 2A of case 2 is covered with a protective coating for the purpose of promoting the sliding of case 2 against an outer surface.

To best advantage, the whole of the outer surface of case 2, i.e. of the surface designed to come into contact with the internal biological tissues, is coated with said protective coating.

Preferentially, the whole of the inner surface of case 2, i.e. of the surface designed to come in contact with the folded-up implant 1, is also covered with the protective coating.

Said coating makes it possible to prevent any aggressive contact by case 2 on the biological tissues, for example mammary, and to thereby promote the passage of case 2 up to the final subcutaneous implantation zone.

Preferentially, the coating has a base of one or more of the materials from the following group:

biocompatible elastomer, of silicone or polyurethane,

paraxylilene, of the PARYLENE® type,

polyvinylpyrrolidone (or PVP),

sodium hyaluronate.

In other words, the coating has the basic function of improving the atraumatic character of case 2.

It is of course conceivable that the coating might be made up of any other biodegradable and biocompatible polymer, without on this account leaving the framework of the invention.

To best advantage, case 2 is fitted with a thread 12 of which a first portion 19 constitutes a fixation seam, said seam forming the locking means 4, and of which a second portion 14, 12A, remaining free, forms the opener member 3, actionable by traction.

The stitch forming seam 19 is chosen to come undone when sufficient traction is exerted on the free portion 14, 12A of thread 12. The stitch is, however, chosen to hold together by itself, and supply an intrinsic interlocking, without it being necessary to exert tension or any other external action on seam 19.

To best advantage, case 2 is provided with a thread 12 having a first portion sewn as a single-thread chain stitch so as to form said locking means 4, and having a second portion 14 that remains free and forms the opener member 3, actionable by traction.

The chain stitch is a sewing stitch well-known as such, which is produced by looping the thread together with itself. Within the framework of the invention, the single thread chain stitch of class 101 within the meaning of standard NF G 05-002 (December 1982) is preferred.

The invention exploits the following altogether specific property of this stitch. This stitch, after the fashion of other sewing stitches, makes it possible to construct a locked assembly. However, if traction is exerted on the free end 12A of the single and continuous thread that served to construct this stitch, and on this condition only, a cascading deconstruction of the stitch will occur, which will result in doing away with the assembly and therefore the locking.

In this way the chain stitch makes it possible, from a single continuous thread, to construct distinct locking means 4 and distinct opener member 3, in particular on account of the manipulations necessary for their respective activation.

To best advantage, the periphery of the lateral opening 6 of sheath 5 is fitted with eyelets 13 designed to be assembled by single-thread chain stitch sewing in order to close said opening 6.

Within the framework of the exemplary embodiment of sheath 5 involving a textile lattice 8 or a fabric, eyelets 13 are delimited by the mesh of lattice 8 (or of the fabric) located in proximity to and along the two opposite edges 8A, 8B designed to be interlocked. In this case, the chain stitches confine, two by two, the weft threads 10 of the opposite edges 8A, 8B as shown in FIG. 2. The chain stitch is thus constructed so that it locks the relative motion of edges 8A, 8B and prevents their spreading or coming apart. However, when traction is exerted on the terminal end 12A of thread 12, then the chain stitch comes undone, which has the effect of doing away with any bond of closure between edges 8A, 8B.

The terminal end 12A corresponds, as shown in FIG. 2, to the left free end of thread 12, when the stitch is carried out from right to left.

To best advantage, as shown in FIG. 1, a chain stitch is carried out both at the level of lattice 8 itself, in order to construct a case 2 in closed configuration, as well as outside of lattice 8 itself, on both sides 14, 18, and in the continuity of the stitch 19 carried out on lattice 8. This arrangement makes it possible to guarantee with surety the interlocking of the two edges 8A, 8B in the case in particular of untimely traction on the free terminal end 12A of thread 12.

Preferentially, as shown in FIG. 5, opener member 3 is endowed with a safety means 21, which enables the prevention, with even greater surety, of any untimely activation of opener member 3.

When said opener member 3 is made up of a terminal portion 14 of thread 12 put together with a chain stitch, the safety means 21 is to best advantage made up of a loop 20 interacting with two chain stitches 21, 22 possibly successive of said terminal portion 14, to block any cascading deconstruction of the chain stitches located downstream, i.e. between said two stitches 21, 22 and seam 19, when the terminal portion 12A is pulled.

Said loop 20 is for example constructed from a segment of thread, two ends of which are linked by a knot 22.

Said loop 20 is preferentially positioned on portion 14 forming opener member 3, so as to be located outside the patient's body when case 2 is positioned inside the patient's body.

In this way, it is enough for the practitioner to undo knot 22 of loop 20, or quite simply to cut the latter, in order to make possible cascading deconstruction of the chain stitches, when traction is exerted on free end 12A.

Portion 14, 12A of thread 12 forming opener member 3 is proportioned in a way that is sufficiently long so that the practitioner can pull on end 12A of the thread from outside, when case 2 is under the patient's skin, in order to carry out the opening of case 2 for the purpose of releasing implant 1. Portion 14 may preferentially be inserted in a catheter, for example a catheter coaxial with, or joined with, the inflation catheter 7.

In the preceding, a chain stitch interacting with eyelets 13 has been described. Of course, the chain stitch can be carried out directly on a non-openwork fabric or on a continuous membrane, for example, without on this account leaving the framework of the invention.

The thread(s) from which locking means 4 is formed can be manufactured form synthetic or natural materials (for example: polyester, polypropylene, cotton, cellulose, silk) according to any construction well known to those skilled in the art (mono- or multifibre, twisted, etc.).

According to an alternative exemplary embodiment of locking means 4, it is possible to conceive, instead and in place of the chain stitch seam, the implementation of a miniaturised “zipper” system, the mobile element of this “zipper” being linked to a traction thread forming the opener members.

According to another alternative exemplary embodiment, locking means 4 can be formed from a “Ziplock®” zipper system, said zipper making possible the opening or closing of case 2, and being to this end linked to a traction thread forming opener member 3.

According to a third alternative exemplary embodiment, locking means 4 is constructed with a “Velcro®” type closure, opener member 3 being formed from a thread arranged to exert a separation force on the complementary “Velcro®” elements, when traction is exerted on said thread.

To best advantage, case 2 is fitted with an optical examination means designed to visualise the inside of the patient's body. The optical examination means may therefore be made up of any endoscopic means well known to those skilled in the art, of the micro-camera sort.

To best advantage, case 2 is also fitted with an illumination means designed to illuminate the inside of the patient's body. The illumination means may be made up of any surgical material well known to those skilled in the art, and based, for example, on fibre optic technology.

In this way, by means of the presence of endoscopic visualisation means attached to case 2, for example on its outer surface 5A, the practitioner can control the implantation operation in a particularly precise way.

To best advantage, case 2 is also fitted, on its outer surface 5A with at least one graduation, and more preferably with a set of graduations, which thus make it possible for the surgeon to determine in real time what length of case 2 has gone into the patient's body during the implantation operation, which facilitates the correct positioning of the implant within the patient's body, and supplements the palpation operations done to this end by the surgeon.

The invention also relates to a manufacturing method for a kit for introduction of a plastic surgery implant into the body of a patient in which:

a plastic surgery implant 1 is supplied or manufactured, said implant 1 presenting a deformable character making it possible for it to pass from a configuration for introduction into the body to a functional configuration within the body,

a case 2 is supplied or manufactured, designed to envelope said implant 1 in the introduction configuration.

To best advantage, said case 2 is provided with an opener member 3, that can be activated by positive action and making it possible for the case to pass from a closed configuration, in which it is capable of confining implant 1 in its introduction configuration, to an open configuration, in which it is capable of making possible the deformation of said implant 1 into its functional configuration.

To best advantage, said method comprises a step for the locking of case 2 in the closed configuration, in which case 2 is provided with a locking means 4 making it possible to immobilise by itself, without any outside action on said means 4, case 2 in the closure configuration, and in which said locking means 4 is functionally linked to opener member 3.

To best advantage, during the locking step, the case is provided with a thread 12 having a first portion sewn as a single-thread chain stitch so as to form said locking means 4, and having a second portion 14 that remains free and forms the opener member 3, actionable by traction.

Preferentially, a case 2 is manufactured that essentially presents, when it is in the closed configuration, a sheath shape with at least one axial opening 5D, 5E at one of the ends 5B, 5C of said sheath 5.

To best advantage, the method in accordance with the invention comprises a step for insertion of implant 1 into sheath 5 in which, as shown in FIG. 4:

implant 1 is shaped in the introduction configuration, in such a way that said implant 1 presents a general shape that is slender overall, of transverse section S,

then, implant 1 is pre-constrained in such a way that it presents an essentially slender shape whose transverse section S has diminished, so as to be less than section D of sheath 5,

then, the pre-constrained implant 1 is introduced into sheath 5 through said at least one axial opening 5E,

then, once sheath 5 envelopes implant 1, the pre-constraint is removed, in such a way that implant 1 returns to its introduction configuration.

In the case of an implant 1 presenting an elastic character, the pre-constraint may consist in exerting longitudinal traction on the implant (as marked by arrows E in FIG. 4). Thus acted upon, implant 1 undergoes a longitudinal extension accompanied by a constriction that leads to reduction of its transverse section S. The implant pre-constrained in this way may then be inserted easily into sheath 5, even if section D of the latter is less than section S of implant 1 in the introduction configuration. Locking means 4 then guarantees that sheath 5 will not open under the effect of the radial centrifugal elastic return force exerted by implant 1, when the pre-constraint is removed.

Thus, sheath 5 can indeed compress implant 1, in such a way as to be able to maximally minimise the transverse dimension of the whole formed by sheath 5 and implant 1.

In a variant of realisation of the method in accordance with the invention, the step for insertion of implant 1 into sheath 5 comprises the following sub-steps:

implant 1 is shaped into the introduction configuration,

then implant 1 is progressively constrained along its length by means of a jig 23, in such a way as to reduce the transverse section S of said implant 1, while simultaneously and progressively covering implant 1 with sheath 5 in the closed configuration.

As shown in FIG. 6, jig 23 preferentially comprises a hollow cylindrical tube, more preferably constructed out of a material presenting a low coefficient of friction with the material making up sheath 5. In the case in which sheath 5 is made out of a textile material, for example a polyester lattice, jig 23 may to best advantage be made out of stainless steel.

Sheath 5 in the closed configuration is threaded by its distal end 5C inside of the hollow tube forming jig 23, while the proximal portion 24 of sheath 5, which is not inside hollow tube 23, is turned around to envelope said tube 3.

We thus have at this stage a hollow tube 23, whose inner and outer walls are at least in part covered and sheathed by sheath 5. One of the openings 23A of hollow tube 23, which are designated in what follows by entry opening, corresponds to that where the transverse section of hollow tube 23 is covered by the flexible bend 26 formed by sheath 5 on tube 23.

Implant 1, which was previously placed in the introduction configuration, is next forcibly introduced into entry opening 23A by its distal end 1A, over a length X sufficient to establish frictional contact between said proximal end 1A and the corresponding zone 27 of sheath 5.

Once this step for priming of implant 1 is carried out, all it then takes is to exert traction on distal end 5C of sheath 5 coaxially in tube 23 to pull implant 1 by friction towards the opening of tube 23 opposite the entry opening 23A.

This displacement of implant 1 is therefore carried out without exerting direct effort on said implant 1A, but simply by using the frictional dragging of implant 1 along the inner surface of sheath 5.

The progression of implant 1 in hollow tube 23 thus makes it possible to unroll, on said implant, the proximal part 24 of sheath 5 which was folded back on the outer surface of hollow tube 23.

Thus, radial compression and covering up of implant 1 are simultaneously achieved, without exerting direct traction effort on the latter, which makes it possible to minimise any risk of deterioration of the implant.

Finally, the invention also relates to use of a chain stitch in accordance with class 101 of standard NF G 05-002 (December 1982) as locking means 4 of a case 2 for introduction of a plastic surgery implant 1 into a patient's body, and in particular of a mammary implant.

The functioning of an introduction kit in accordance with the invention will now be described.

Initially, the surgeon has in his possession a kit in which implant 1 is enveloped by a case 2. The proximal end of case 2 is of one piece with a catheter 7 used to effectuate both the opening of case 2 in the patient's body and the inflation of implant 1.

The course of the operation is then the following.

The surgeon introduces the total unit formed from implant 1 and case 2 under the patient's skin, by the distal end 5C, by passing through a previously made incision.

The practitioner makes the kit move forward by pushing the latter up to the final zone of positioning of implant 1, under the patient's skin.

The relatively rigid character of the total unit of implant/case in the axial direction is used by the surgeon to spread the tissues apart and prepare a housing for the implant. In this way, the surgeon avoids prior use of a tissue retractor.

This same axially rigid but flexible character of the total unit of implant/case makes possible, for the surgeon, an easy and rapid introduction of said total unit under the patient's skin.

The practitioner then takes hold of the terminal end 12A of thread 12, which is coming out of the distal end of catheter 7, and exerts traction on said thread 12 so as to undo the chain stitch seam 19 forming locking means 4. Thus unsewn, sheath 5 has the tendency to naturally resume a plane shape, especially if it presents shape memory properties.

The practitioner then conducts the swelling of implant 1, if this presents an inflatable character, by insufflating an inflation fluid through catheter 7. Once the implant is inflated, the practitioner gives an impulse to catheter 7 in such a way that implant 1 comes apart from catheter 7 (de-retracting).

The practitioner then withdraws catheter 7 outside of the patient's body, to which catheter case 2 is attached, for example by gluing. Implant 1 is thus arranged within the patient's body, and can fulfil its therapeutic and/or aesthetic function.

The practitioner may also, in another variant of realisation, conduct explantation of case 2 before inflation of implant 1.

It is to be noted finally that case 2 in accordance with the invention can be used for introduction into the body of any other prosthetic device, and that its usage is consequently not limited to subcutaneous implantation of plastic surgery implants.

The invention finds its industrial application in the manufacture of plastic surgery implants.

1. A kit for introduction of a plastic surgery implant into the body of a patient comprising:
(a) a plastic surgery implant designed to be implanted in the body of a patient, said implant presenting a deformable character which makes it possible for it to pass from a configuration for introduction into the body to a functional configuration within the body,
(b) a case shaped to envelope said implant in the introduction configuration, said case being provided with an opener member that can be activated by positive action making it possible for it to pass on the one hand from a closed configuration, in which it confines implant in its introduction configuration to, on the other hand, an open configuration, in which it enables deformation of said implant into its functional configuration,
said case comprising a locking means, linked functionally to the opener member and making it possible to immobilise by itself, without any external action on said means, case in the closure configuration, wherein said case is provided with a thread having a first portion sewn as a single-thread chain stitch so as to form said locking means, and having a second portion that remains free and forms the opener member, actionable by traction.

2. The kit of claim 1, wherein said case comprises a sheath fitted with at least one lateral opening arranged along its length, said opening being closed by said locking means when case is in the closed configuration and said opening being cleared, to make possible deformation of implant into its functional configuration, when case is in the open configuration.

3. The kit of claim 2, wherein said sheath presents an essentially tubular shape and is split over all or part of its length, said split making up lateral opening.

4. The kit of claim 2, wherein said sheath is constructed from a flexible material.

5. The kit of claim 2, wherein said sheath is formed from a fabric whose two opposite edges (8A, 8B) are interlocked by locking means, such that the fabric is shaped in an essentially tubular way.

6. The kit of claim 5, wherein said fabric is constructed by weaving of threads of material principally based on polyamide, of the nylon® thread type.

7. The kit of claim 2, wherein the periphery of lateral opening is fitted with eyelets, designed to be assembled by single-thread chain stitch sewing in order to close said opening.

8. The kit of claim 7, wherein said eyelets are delimited by the mesh of the fabric located in proximity to and along said edges.

9. The kit of claim 1, wherein said chain stitch belongs to class 101 of standard NF G 05-002 of December 1982.

10. The kit of claims 1, wherein said implant comprises at least one flexible pouch defining a predetermined internal volume, said at least one flexible pouch being fitted with a connection means constructed to receive a connection unit designed to be linked to a fluid source, for the purpose of effectuating expansion of said pouch inside the patient's body by filling with the fluid.

11. The kit of claim 1, wherein said case is fitted with an optical examination means designed to visualise the inside of the patient's body and/or with an illumination means designed to illuminate the inside of the patient's body.

12. The kit of claim 1, wherein said case is fitted with at least one graduation on its outer surface.

13. The kit of claim 1, wherein said at least a part of surface of case is covered with a coating for the purpose of promoting the sliding of case against an outer surface.

14. The kit of claim 13, wherein said coating is based on at least one materials selected from group comprising
biocompatible elastomer, of the silicone or polyurethane type,
paraxylilene, of the parylene® sort,
polyvinylpyrrolidone, and
sodium hyaluronate.

15. The kit of claim 1, wherein said implant is selected from the group consisting of:
mammary implants,
pectoral implants,
leg implants,
arm implants, and
buttocks implants.

16. A case for introduction of a plastic surgery implant into the body of a patient, said implant presenting a deformable character making it possible for it to pass from a configuration for introduction into the body to a functional configuration within the body, comprising: as case being shaped to envelope said implant in the introduction configuration and being provided with an opener member that can be activated by positive action making it possible for it to pass, on the one hand, from a closed configuration, in which it confines implant in its introduction configuration to, on the other hand, an open configuration, in which it enables deformation of said implant into its functional configuration, said case comprising a locking means linked functionally to opener member and making it possible to immobilise by itself, without any external action on said means, case in the closure configuration, said case wherein it is provided with a thread having a first portion sewn as a single-thread chain stitch so as to form said locking means, and having a second portion (11)that remains free and forms the opener member, actionable by traction.

17. The case of claim 16, wherein it comprises a sheath fitted with at least one lateral opening arranged along its length, said lateral opening being closed by said locking means when case is in the closed configuration and said opening being cleared, in order to make possible the deformation of said implant into its functional configuration, when case is in the open configuration.

18. The case of claim 17, wherein said sheath has an essentially tubular shape and is split over all or part of its length, said split making up said lateral opening.

19. The case of claim 18, wherein said sheath is formed from a fabric, the two opposite edges of which are interlocked by a locking means, in such a way that said fabric is shaped in a generally tubular way.

20. The case of claim 19, wherein said fabric is constructed by weaving of threads principally based on polyamide, of the nylon® thread type.

21. The case of claim 18, wherein the periphery of lateral opening is fitted with at least one eyelet, designed to be assembled by single-thread chain stitch sewing in order to close said opening.

22. The case of claim 21, wherein said eyelets are formed by meshes of the fabric located in proximity to and along said edges.

23. The cases of claim 16, wherein said case is made from a flexible material.

24. The case of claim 16, wherein said case is constructed from an elastic material.

25. The case of claim 16, wherein said at least one part of the surface of said case is covered with a coating for the purpose of promoting the sliding of case against an outer surface.

26. The case of claim 25, wherein said coating has a base of at least one materials selected from among the group consisting of:
biocompatible elastomer, of the silicone or polyurethane type,
paraxylilene, of the parylene® type,
polyvinylpyrrolidone, and
sodium hyaluronate.

27. The case of claim 16, wherein said chain stitch belongs to class 101 of standard NF G 05-002 of December 1982.

28. The case of claim 16, wherein said case is fitted with an optical examination means designed to visualise the inside of the patient's body and/or with an illumination means designed to illuminate the inside of the patient's body.

29. The case of claim 16, wherein said case is fitted with at least one graduation on its outer surface.

30. The case of claim 16 for introduction of a plastic surgery implant into the body of a patient, wherein said implant is selected from the group consisting of:
mammary implant,
pectoral implant,
leg implant,
arm implant, and
buttocks implant.

31. A manufacturing method for a kit for introduction of a plastic surgery implant into the body of a patient, comprising:
providing a plastic surgery implant, said implant presenting a deformable character that makes it possible for it to pass from a configuration for introduction into the body to a functional configuration within the body;
providing a case, designed to envelope said implant in the introduction configuration, said case essentially presenting, when it is in the closed configuration, a sheath shape,
inserting said implant into a sheath in which;
said implant is shaped in the introduction configuration; and
progressively constraining said implant along its length by means of a jig, so as to reduce the transverse section of said implant, while simultaneously covering implant with sheath in the closed configuration.

32. The method of claim 31, further comprising a step providing said case with an opener member that can be activated in order to make it possible for said case to pass from a closed configuration, in which it is capable of confining said implant in its introduction configuration, to an open configuration, in which it is capable of enabling the deformation of said implant into its functional configuration, said method comprising a step for locking of said case in the closed configuration in which said case is provided with a locking means making it possible for it to immobilise by itself, without any external action on said means, case in the closure configuration, and in which said locking means is functionally linked to the opener member.

33. The method of claim 32, wherein during said locking step, said case is provided with a thread having a first portion sewn as a single-thread chain stitch so as to form said locking means, and having a second portion that remains free and forms the opener member, actionable by traction.

34. The use of a chain stitch in accordance with class 101 of standard NF G 05-002 of December 1982 as a locking means of a case for introduction of a plastic surgery implant into the body of a patient.

35. The kit of claim 3, wherein said sheath is constructed from a flexible material.
